(19) United States
(12) Patent Application Publication
(10) Pub. No.: US 2010/0161008 A1
(43) Pub. Date: Jun. 24, 2010

(54) Potential therapy apparatus and combined electric therapy apparatus

(75) Inventors: Mikihiko Hase, Tokyo (JP); Masakazu Karita, Tokyo (JP)

Correspondence Address: POSZ LAW GROUP, PLC, 12040 SOUTH LAKES DRIVE, SUITE 101, RESTON, VA, 20191, US

(73) Assignee: COCOROCA CORPORATION, Tokyo (JP)

(21) Appl. No.: 12/310,478

(22) PCT Filed: Aug. 31, 2007
(86) PCT No.: PCT/JP2007/067058

§ 371 Date: Apr. 21, 2009

(30) Foreign Application Priority Data
Sep. 1, 2006 (JP) 2006-237760

(51) Int. Cl. A61N 1/36

(52) U.S. Cl. 607/72; 607/3

(57) ABSTRACT

An electric therapy apparatus 1 of the present invention includes: a voltage generating unit 14 for generating a predetermined voltage; a control unit 2 for varying the voltage generated by the voltage generating unit 14 and the output time of the voltage according to a 1/f fluctuation cycle; and potential therapy conductor terminals 27, 28 for applying the potential from the voltage generating unit 14 to a therapy object, and as a result, the therapy object is prevented from getting acclimatized to stimulation by a constant potential, thereby allowing the human body to be imparted with a relaxing feeling, while sustaining the therapeutic effect of the apparatus.

### TECHNICAL FIELD

The present invention relates to a potential therapy apparatus for therapeutic treatment of the human body through application of potential, and to a combined electric therapy apparatus that allows carrying out potential therapy and low-frequency therapy.

### BACKGROUND ART

Known therapy apparatuses include potential therapy apparatuses for treating headaches, shoulder stiffness, chronic constipation, insomnia and the like through application of AC high voltage potential to part or the entirety of the human body, and low-frequency therapy apparatuses for treating shoulder stiffness, muscle fatigue and the like through application of low-frequency current to part or the entirety of the human body.

For instance, in a potential therapy apparatus 100 such as the one illustrated in FIG. 6, voltage supplied from an AC 100 V power source 101 to a transformer 102 via a switch SW and converted to high voltage by the transformer 102, is made positive/negative asymmetrical by three resistors R₁, R₂, R₃ and a diode D, and is applied to an electric bed 103, whereupon the electric bed 103 applies potential to the human body (Patent document 1). In this potential therapy apparatus 100, a switch SW switches the primary tap of the transformer 102 to adjust thereby the output voltage.

For instance, voltages of 3000 V, 6000 V and 9000 V are ordinarily set as the output voltage. In the potential therapy apparatus 100 having the above configuration, a constant potential set by the switch SW is applied to the human body. This is problematic in that, when being applied a constant potential like this, the human body gets acclimatized to that potential, and the therapeutic effect becomes weaker as a result. Accordingly, various potential therapy apparatuses have been proposed in which the output voltage is caused to vary in such a way so as to prevent a constant potential from being applied to the body.

Patent document 1: Japanese Unexamined Patent Application Publication S58-146361

### DISCLOSURE OF THE INVENTION

### Problem to be Solved by the Invention

However, output voltage changes in such potential therapy apparatuses are based on uniform patterns. This is problematic in that, in consequence, the subject may fail to respond to the changes, or may experience some mood disorder during or after the therapy, as a result of which the therapy cannot be conducted comfortably.

In this context, it is known that “1/f fluctuations”, which constitute a certain rhythm that is encountered in nature, for instance in water, wind and light, has a relaxing effect on human beings. That is presumably so because the rhythms of the human body, as can be observed in, for instance, heartbeat rhythms, are also close to such “1/f fluctuations”. Even if the output voltage of the potential therapy apparatus is made to vary, therefore, it is thought that a relaxing therapy may fail to be achieved if the rhythms of the human body and changes in output voltage are mismatched, in which case the mismatch precludes enhancing the therapeutic effect.

In the light of the above, it is an object of the present invention to provide a potential therapy apparatus and a combined electric therapy apparatus that allow preventing a therapy object from getting acclimatized to stimulation by a constant potential or constant current, that allow imparting a relaxing feeling on humans, and that allow sustaining/enhancing the therapeutic effect.

### Means for Solving the Problem

In order to achieve the above object, the present invention provides a potential therapy apparatus, comprising: a voltage generating unit for generating a predetermined voltage; a control unit for varying, according to a 1/f fluctuation cycle, a voltage generated by the voltage generating unit and varying the output time of the voltage; and a potential therapy conductor terminal for applying potential from the voltage generating unit to a therapy object (Invention 1).

The voltage generating unit may be for instance a transformer or the like, but is not limited thereto.

The voltage control unit can be, although not limited thereto, a combination of, for instance, a CPU (central processing unit) of a computer, an LPF (low pass filter), a switch for switching the tap of an amplifier or a transformer.

The potential therapy conductor terminal may be an electric bed for applying potential to the entire therapy object (human body), or a pen-type or similar local conductor terminal for applying potential to part of the therapy object (human body).

The above invention (Invention 1) allows varying the potential applied to the therapy object (human body) according to a 1/f fluctuation cycle, and allows varying the output time of the potential applied to the therapy object (human body) also according to a 1/f fluctuation cycle. Therefore, the invention allows preventing the therapy object from getting acclimatized to potential stimulation, allows bringing stimulation changes closer to the rhythms of the human body, and allows therapy to be carried out while imparting a relaxing feeling to the user. The invention allows thereby sustaining/enhancing the therapeutic effect.

Preferably, the above invention (Invention 1) further comprises an output operation unit that can set an upper limit of the output voltage, wherein the control unit causes the voltage to vary within the upper limit of output voltage set by the output operation unit according to a 1/f fluctuation cycle (Invention 2).

The above invention (Invention 2) allows varying the potential applied to the therapy object (user), according to a 1/f fluctuation cycle, within an output range as desired by the therapy object, and allows also varying the output time of the potential according to a 1/f fluctuation cycle, within that output range.

In the above invention (Invention 1 or 2), preferably, the control unit comprises a storage unit in which voltage control data for varying the voltage generated by the voltage generating unit, and time control data for varying the output time of the voltage generated by the voltage generating unit are associated with each other and then stored; and a processing unit for varying the voltage generated by the voltage generating unit and varying the output time of the voltage according to a 1/f fluctuation cycle, based on voltage control data and time control data stored in the storage unit (Invention 3).

In the above invention (Invention 3), voltage and output time are caused to vary according to a 1/f fluctuation cycle on the basis of voltage control data and time control data. As a result, this allows applying potential to the therapy object without burdening the control unit.

Preferably, the above invention (Invention 3), further comprises a timer for controlling energization time, and a timer operation unit that can set an energization time, wherein the processing unit causes the voltage generated by the voltage generating unit, and the output time of the voltage, to vary according to a 1/f fluctuation cycle, until termination of the energization time set by the timer operation unit (Invention 4).

The above invention (Invention 4) allows the control unit to vary voltage and output time continuously, without breaks, according to a 1/f fluctuation cycle, during the energization time set by the timer operation unit.

Upon termination of the energization time set by the timer operation unit in the above invention (Invention 4), preferably, the storage unit stores information relating to a termination point in the voltage control data and the time control data at the time at which the energization time terminates, when information relating to the termination point is stored in the storage unit, the control unit causes the voltage generated by the voltage generating unit, and the output time of the voltage, to vary according to a 1/f fluctuation cycle, based on the voltage control data and the time control data from the termination point onward, in accordance with information relating to the termination point (Invention 5).

Depending on the energization time set by the timer operation unit, energization may terminate halfway through the voltage control data and time control data. In the above invention (invention 5), however, when therapy terminates halfway through voltage control data and time control data having for instance a length of 20 minutes (for instance, after 15 minutes), the potential applied to the therapy object when therapy is resumed again is based on voltage control data and time control data after therapy termination (from minute 15 onwards). As a result, the potential variation pattern is different for each therapy round, which allows preventing the therapy object from getting acclimatized to potential stimulation.

In the above inventions (Inventions 1 to 5), preferably, the control unit increases and reduces gradually the voltage generated by the voltage generating unit (with, for instance, a 1000 V rise and fall over 0.5 seconds) (Invention 6).

For instance, a high voltage of 9000 V applied suddenly gives rise to a large inrush current that may overload circuits and may result in excessive stimulation of the body. In the above invention (Invention 6), however, inrush currents are made small by increasing and reducing the voltage gradually, which allows diminishing adverse effects on the circuits and the body.

Preferably, the above inventions (Inventions 1 to 6) further comprise a plurality of connectors into which respective plugs of a plurality of the potential therapy conductor terminals are inserted; and a sensor that detects the insertion of the plugs into the plurality of connectors respectively and sends a detection signal to the control unit, and when the plugs are inserted into two or more connectors of the plurality of connectors, the control unit outputs an indication signal on the basis of a detection signal from the sensor (Invention 7).

The potential therapy apparatus cannot be operated when plugs of potential therapy conductor terminals are inserted into two or more connectors. Herein, the above invention (Invention 7) allows outputting an indication signal urging the user to select one of the potential therapy conductor terminals being used, when plugs of potential therapy conductor terminals are inserted into two or more connectors. As a result, the potential therapy apparatus is prevented from operating when plugs of potential therapy conductor terminals are inserted into two or more connectors.

The present invention provides also a combined electric therapy apparatus that comprises a voltage generating unit for generating a predetermined voltage; a pulse voltage generating unit for generating a predetermined pulse voltage; a control unit for controlling the voltage generating unit and the pulse voltage generating unit; a potential therapy conductor terminal for applying potential from the voltage generating unit to a therapy object; and a low-frequency therapy conductor terminal for applying pulse current from the pulse voltage generating unit to a therapy object; wherein the control unit causes the voltage generated by the voltage generating unit, and the output time of the voltage, to vary according to a 1/f fluctuation cycle, and causes the pulse height, pulse width and pause time of the pulse voltage generated by the pulse voltage generating unit to vary according to a 1/f fluctuation cycle (Invention 8).

The above invention (Invention 8) is thus a combined electric therapy apparatus capable of performing two therapies, namely potential therapy and low-frequency therapy. When potential therapy is carried out, the combined electric therapy apparatus allows varying voltage and the output time of the voltage according to a 1/f fluctuation cycle, while when low-frequency therapy is carried out, the apparatus allows varying the pulse height, pulse width and pause time of pulse voltage according to a 1/f fluctuation cycle. As a result, the pulse frequency can be made to vary also according to a 1/f fluctuation cycle. This allows preventing the therapy object from getting acclimatized to potential stimulation or current stimulation during potential therapy or low-frequency therapy, and allows imparting a relaxing feeling on the user by bringing stimulation changes closer to the rhythms of the human body. The invention allows thereby sustaining/enhancing the therapeutic effect.

Preferably, the above invention (Invention 8) further comprises an output operation unit that can set an upper limit of output voltage or of output pulse voltage, wherein the control unit causes, according to a 1/f fluctuation cycle, the voltage or pulse height to vary within the upper limit of output voltage or output pulse voltage set by the output operation unit (Invention 9).

The above invention (Invention 9) allows varying the potential or pulse voltage applied to the therapy object (user), according to a 1/f fluctuation cycle, within an output range as desired by the therapy object, and allows also varying the output time of the potential, or the pulse width and pause time of the pulse voltage, according to a 1/f fluctuation cycle, within that output range.

In the above inventions (Inventions 8 and 9), preferably, the control unit comprises: a storage unit in which voltage control data for varying the voltage generated by the voltage generating unit, and time control data for varying the output time of the voltage generated by the voltage generating unit are associated with each other and then stored, and pulse height control data for varying pulse height of the pulse voltage generated by the pulse voltage generating unit, pulse width control data for varying the pulse width of the pulse voltage generated by the pulse voltage generating unit, and pause time control data for varying the pause time of the pulse voltage generated by the pulse voltage generating unit are associated with each other and then stored; and a processing unit for varying the voltage generated by the voltage generating unit and varying the output time of the voltage according to a 1/f fluctuation cycle, based on voltage control data and time control data stored in the storage unit, and varying according to a 1/f fluctuation cycle the pulse height, pulse width and pause time of the pulse voltage generated by the pulse voltage generating unit on the basis of pulse height control data, pulse width control data and pause time control data stored in the storage unit (Invention 10).

The above invention (Invention 10) allows varying voltage and output time according to a 1/f fluctuation cycle on the basis of voltage control data and time control data, and allows varying pulse height, pulse width and pause time according to a 1/f fluctuation cycle on the basis of pulse height control data, pulse width control data and pause time control data. This allows applying potential or pulse voltage to the therapy object without burdening the control unit.

Preferably, the above invention (Invention 10) further comprises a timer for controlling energization time, and a timer operation unit that can set an energization time, wherein the processing unit causes the voltage generated by the voltage generating unit, and the output time of the voltage, to vary according to a 1/f fluctuation cycle, and causes the pulse height, pulse width and pause time of the pulse voltage generated by the pulse voltage generating unit to vary according to a 1/f fluctuation cycle, until termination of the energization time set by the timer operation unit (Invention 11).

The above invention (Invention 11) allows the control unit to cause voltage and output time to vary according to a 1/f fluctuation cycle, and to cause pulse height, pulse width and pause time to vary according to a 1/f fluctuation cycle, continuously and without breaks, during the energization time set by the timer operation unit.

Upon termination of the energization time set by the timer operation unit in the above invention (Invention 11), preferably, the storage unit stores information relating to a first termination point in the voltage control data and the time control data at the time at which the energization time terminates, and stores information relating to a second termination point in the pulse height control data, the pulse width control data and the pause time control data at the time at which the energization time terminates, and when information relating to the first termination point is stored in the storage unit, the processing unit causes the voltage generated by the voltage generating unit, and the output time of the voltage, to vary according to a 1/f fluctuation cycle, based on the voltage control data and the time control data, from the first termination point onward, in accordance with information relating to the first termination point, and when information relating to the second termination point is stored in the storage unit, the processing unit causes the pulse height, pulse width and pause time of the pulse voltage generated by the pulse voltage generating unit to vary according to a 1/f fluctuation cycle, based on the pulse height control data, the pulse width control data and the pause time control data, from the second termination point onward (Invention 12).

Depending on the energization time set by the timer operation unit, energization may terminate halfway through the voltage control data and time control data, or halfway through the pulse height control data, pulse width control data or pause time control data. In the above invention (Invention 12), however, when therapy terminates halfway through voltage control data and time control data, or halfway through pulse height control data, pulse width control data and pause time control data having for instance a length of 20 minutes (for instance, after 15 minutes), the potential or the pulse voltage applied to the therapy object when therapy is resumed again is based on voltage control data and time control data or on pulse height control data, pulse width control data and pause time control data after therapy termination (from minute 15 onwards). This allows preventing the therapy object from getting acclimatized to potential stimulation or pulse voltage stimulation.

In the above inventions (Inventions 8 to 12), preferably, the control unit increases and reduces gradually the voltage generated by the voltage generating unit (Invention 13). For instance, a high voltage of 9000 V applied suddenly gives rise to a large inrush current that may overload circuits and may result in excessive stimulation of the body. In the above invention (Invention 13), however, inrush currents are made small by increasing and reducing the voltage gradually, which allows diminishing adverse effects on the circuits and the body.

Preferably, the above inventions (Inventions 8 to 13) further comprise a first connector into which a plug of the potential therapy conductor terminal can be inserted; a second connector into which a plug of a low-frequency therapy conductor terminal can be inserted; a first sensor for detecting that a plug of the potential therapy conductor terminal is inserted into the first connector, and for sending a first detection signal to the control unit; and a second sensor for detecting that a plug of the low-frequency therapy conductor terminal is inserted into the second connector, and for sending a second detection signal to the control unit; such that, upon receiving the first detection signal, the control unit controls the voltage generating unit, and upon receiving the second detection signal, the control unit controls the pulse voltage generating unit (Invention 14).

In the above invention (Invention 14) the plug of the potential therapy conductor terminal need only be inserted into the first connector to carry out potential therapy, and the plug of the low-frequency therapy conductor terminal need only be inserted into the second connector to carry out low-frequency therapy. The user can therefore omit the operation of selecting the therapy method.

Preferably, the above invention (Invention 14) further comprises a liquid crystal display, wherein upon receiving the first detection signal or the second detection signal, the control unit displays on the liquid crystal display a therapy setting display image based on the first detection signal or second detection signal (Invention 15).

In the above invention (Invention 15), for instance a therapy setting display image relating to potential therapy is displayed on the liquid crystal display when the control unit detects a first detection signal, while a therapy setting display image relating to low-frequency therapy is displayed on the liquid crystal display when the control unit detects a second detection signal. As a result, the user can select the therapy setting of the desired therapy method simply by inserting the plug of the therapy conductor terminal into a connector.

When in the above invention (Invention 14 or 15) the control unit in a state of being receiving the first detection signal or the second detection signal outputs an indication signal upon receiving the second detection signal or the first detection signal (Invention 16).

In electric therapy apparatuses capable of carrying out plural therapy methods such as potential therapy, low-frequency therapy and the like, plural therapy methods cannot be carried out simultaneously in one single therapy round. The above invention (Invention 16) allows outputting an indication signal urging the user to select a therapy method, even when both the plug of the potential therapy conductor terminal and the plug of the low-frequency therapy conductor terminal are simultaneously inserted into connectors.

### ADVANTAGEOUS EFFECT OF THE INVENTION

The present invention succeeds thus in providing a potential therapy apparatus and a combined electric therapy apparatus that allow preventing a therapy object from getting acclimatized to stimulation by constant potential, that allow imparting a relaxing feeling to the human body, and that allow the therapeutic effect to be sustained.

### BRIEF DESCRIPTION OF THE DRAWINGS

FIG. 1 is a circuit block diagram illustrating a combined electric therapy apparatus according to an embodiment of the present invention;

FIG. 2 is a block diagram illustrating a control unit in a combined electric therapy apparatus according to an embodiment of the present invention;

FIG. 3 is a flowchart illustrating a process operation of potential therapy being carried out using an electric bed in a combined electric therapy apparatus according to an embodiment of the present invention;

FIG. 4 is a flowchart illustrating an error display process in a combined electric therapy apparatus according to an embodiment of the present invention;

FIG. 5 is a flowchart illustrating a process operation of low-frequency therapy being carried out using a low-frequency therapy probe in a combined electric therapy apparatus according to an embodiment of the present invention; and

FIG. 6 is a circuit block diagram illustrating a conventional potential therapy apparatus.

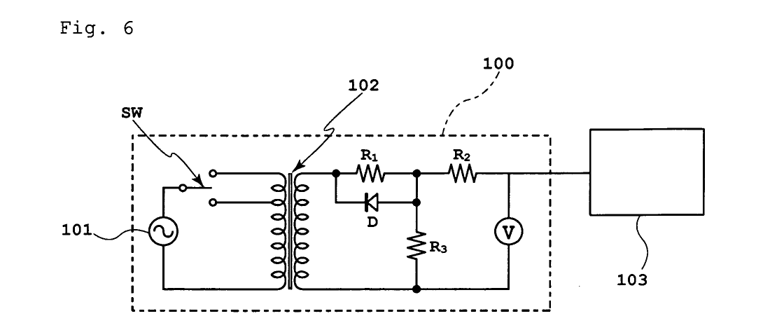

### DESCRIPTION OF THE REFERENCE NUMERALS

1 . . . combined electric therapy apparatus
2 . . . control unit
   201 . . . CPU (processing unit)
   202 . . . main storage unit (storage unit)
   203 . . . sub storage unit (storage unit)
3 . . . operating unit
4 . . . timer
6 . . . sound IC
61 . . . speaker
7 . . . image IC
71 . . . liquid crystal display
8, 9 . . . sine wave generator
10, 11, 20 . . . variable resistor
12, 23 . . . amplifier
13, 22 . . . switch
14, 15, 24 . . . transformer (voltage generating unit, pulse voltage generating unit)
16, 17, 25 . . . connector
18, 19, 26 . . . sensor
21 . . . pulse wave generating circuit
27 . . . electric bed (potential therapy conductor terminal)
28 . . . potential therapy probe (potential therapy conductor terminal)
29 . . . low-frequency therapy probe (low-frequency therapy conductor terminal)

### BEST MODE FOR CARRYING OUT THE INVENTION

A combined electric therapy apparatus according to an embodiment of the present invention is explained next with reference to accompanying drawings.

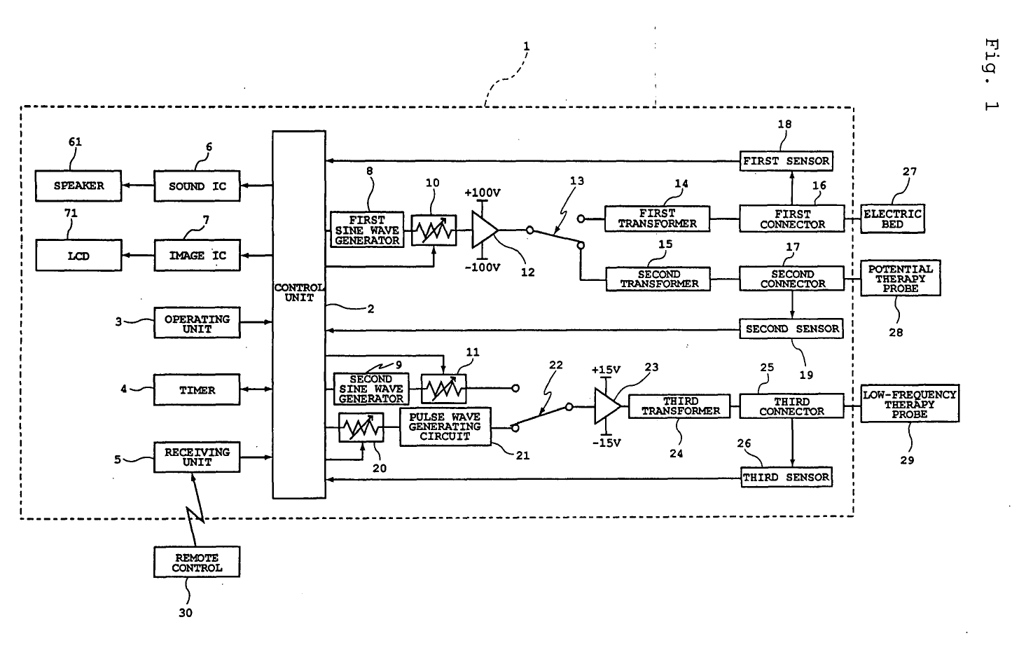

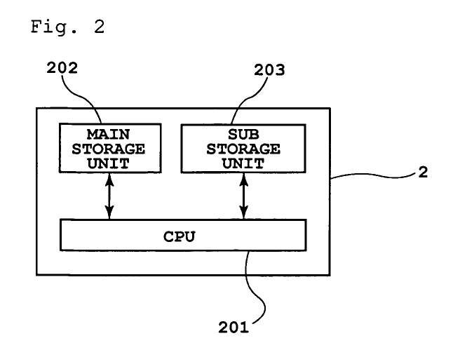

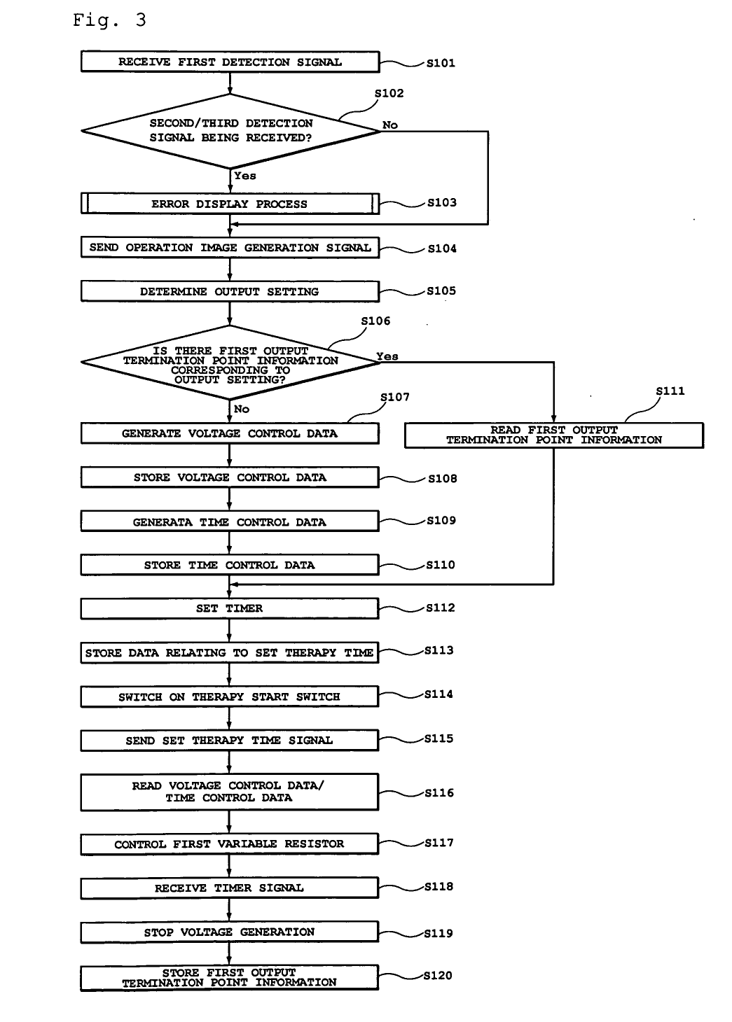

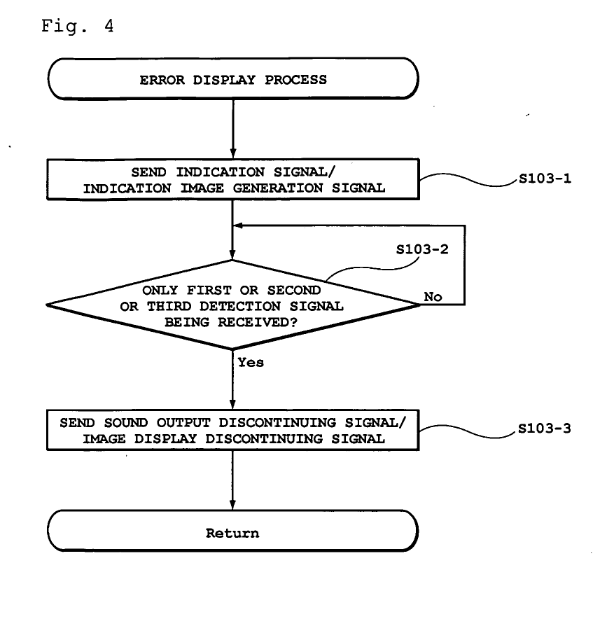

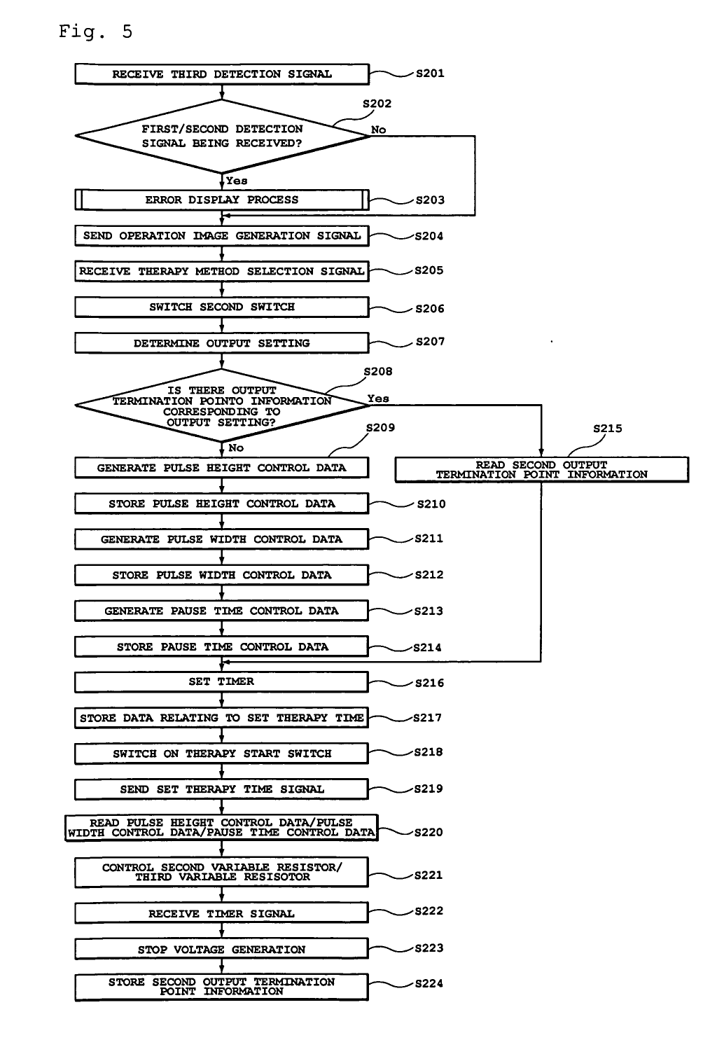

FIG. 1 is a circuit block diagram illustrating a combined electric therapy apparatus according to the present embodiment; FIG. 2 is a block diagram illustrating a control unit of the combined electric therapy apparatus according to the present embodiment; FIG. 3 is a flowchart illustrating a process operation of potential therapy in the combined electric therapy apparatus according to the present embodiment; FIG. 4 is a flowchart illustrating a process operation of error display in the combined electric therapy apparatus according to the present embodiment; and FIG. 5 is a flowchart illustrating the process operation of low-frequency therapy in the combined electric therapy apparatus according to the present embodiment.

As illustrated in FIG. 1, a combined electric therapy apparatus 1 according to the present embodiment comprises a control unit 2, an operating unit 3 connected to the control unit 2, a timer 4, a receiving unit 5, a sound IC 6 and an image IC 7, a speaker 61 connected to the sound IC 6, a liquid crystal display (LCD) 71 connected to the image IC 7, a first sine wave generator (LPF, low pass filter) 8 and a second sine wave generator 9 connected to the control unit 2, a first variable resistor 10 connected to the first sine wave generator 8 and the control unit 2, a second variable resistor 11 connected to the second sine wave generator 9 and the control unit 2, a first amplifier 12 connected to the control unit 2 via the first sine wave generator 8 and the first variable resistor 10, a first transformer 14 and a second transformer 15 connected to the first amplifier 12 via a first switch 13, a first connector 16 and a second connector 17 connected respectively to the first transformer 14 and the second transformer 15, a first sensor 18 and a second sensor 19 connected respectively to the first connector 16 and the second connector 17, a third variable resistor 20 connected to the control unit 2, a pulse wave generating circuit 21 connected to the control unit 2 via the third variable resistor 20, a second amplifier 23 connected to the second variable resistor 11 and the pulse wave generating circuit 21 via a second switch 22, a third transformer 24 connected to the second amplifier 23, a third connector 25 connected to the third transformer 24, and a third sensor 27 connected to the third connector 26.

As illustrated in FIG. 2, the control unit 2 comprises a CPU 201, a main storage unit 202 and a sub storage unit 203. The main storage unit 201 stores, for instance, a program for running the combined electric therapy apparatus 1, and a 1/f fluctuation conversion program that can generate various control data according to which output voltage, output time of the output voltage, as well as pulse height, pulse width and pause time (time from pulse fall to pulse rise) are caused to vary according to a 1/f fluctuation cycle.

The CPU 201 performs input and output of signals between the various constituent elements provided in the combined electric therapy apparatus 1, in accordance with a program stored in the main storage unit 202. Also, the CPU 201 generates a voltage control data for converting an output voltage on the basis of a 1/f fluctuation cycle and a time control data for converting an output time of a voltage on the basis of a 1/f fluctuation cycle, in accordance with a 1/f fluctuation conversion program stored in the main storage unit 202. The output voltage control data comprising the voltage control data and the time control data is 20-minute output voltage control data, according to output settings, but are not limited thereto. The data may also be 40-minute output voltage control data or 60-minute output voltage control data. Habituation of the human body to stimulation changes is prevented by controlling and changing voltage and output time on the basis of at least 20-minute output voltage control data. The output voltage control data has data sequences, with data sequence numbers 1 to 256, relating to output voltage and output time. On the basis of the output voltage control data, the CPU 201 repeatedly executes a process in which the CPU 201 outputs a predetermined voltage for a predetermined time, sequentially from the data sequence number 1 of the output voltage control data, so that after outputting a predetermined voltage for a predetermined time on the basis of the data of number 256, the CPU 201 returns to the number 1. Although in the present embodiment the output voltage control data has data sequences relating to output voltage and output time, with data sequence numbers 1 to 256, the output voltage control data is not limited thereto. For instance, the output voltage control data may have data sequences relating to output voltage and output time, with data sequence numbers 1 to 1024, or may have data sequences relating to output voltage and output time, with data sequence numbers 1 to 4096.

The CPU 201 generates pulse height control data for converting pulse height according to a 1/f fluctuation cycle, pulse width control data for converting pulse width according to a 1/f fluctuation cycle and pause time control data for converting pause time according to a 1/f fluctuation cycle, in accordance with a 1/f fluctuation conversion program stored in the main storage unit 202. The pulse voltage control data comprising the pulse height control data, the pulse width control data and the pause time control data is 20-minute pulse voltage control data, according to output settings, but is not limited thereto. The pulse voltage control data may also be 40-minute pulse voltage control data or 60-minute pulse voltage control data. Habituation of the human body to stimulation changes is prevented by controlling and changing the pulse height, the pulse width and the pause time on the basis of at least 20-minute pulse voltage control data. The pulse voltage control data has data sequences, with data sequence numbers 1 to 256, relating to pulse height, pulse width and pause time. On the basis of the pulse voltage control data, the CPU 201 repeatedly executes a process in which the CPU 201 controls the pulse voltage, sequentially from the data sequence number 1 of the pulse voltage control data, and controls the pulse voltage on the basis of number 256 data, after which the CPU 201 returns to number 1. Although in the present embodiment the pulse voltage control data has data sequences relating to pulse height, pulse width and pause time, with data sequence numbers 1 to 256, the control data is not limited thereto. For instance, the control data may have data sequences relating to pulse height, pulse width and pause time, with data sequence numbers 1 to 1024, or may have data sequences relating to pulse height, pulse width and pause time, with data sequence numbers 1 to 4096.

The CPU 201 controls the first variable resistor 10 on the basis of voltage control data and time control data, and controls the second variable resistor 11 and the third variable resistor 20 on the basis of pulse height control data, pulse width control data and pause time control data.

The sub storage unit 203, which comprises a nonvolatile memory or the like, associates and stores voltage control data and time control data generated by the CPU 201, and stores information relating to the point at which output of potential terminates (first output termination point information), on the basis of voltage control data and time control data. The sub storage unit 203 associates and stores pulse height control data, pulse width control data and pause time control data generated by the CPU 201, and stores information relating to the point at which output of pulse voltage terminates (second output termination point information), on the basis of pulse height control data, pulse width control data and pause time control data.

The first sine wave generator 8 and the second sine wave generator 9 generate sine waves of predetermined frequency on the basis of control signals from the control unit 2.

The first variable resistor 10, the second variable resistor 11 and the third variable resistor 20 adjust the output voltage on the basis of control signals from the control unit 2 (CPU 201). The first variable resistor 10 adjusts a sine wave generated by the first sine wave generator 8 to a predetermined output voltage. The second variable resistor 11 adjusts a sine wave generated by the second sine wave generator 9 to a predetermined output voltage, and generates a predetermined pulse wave. The third variable resistor 20 performs adjustment to a predetermined output voltage, on the basis control signals from the control unit 2 (CPU 201). The first variable resistor 10 adjusts output voltage to 0 to 15 V, the second variable resistor 11 adjusts output voltage to 0 to 20 V, and the third variable resistor 20 adjusts output voltage to 0 to 20 V.

The first amplifier 12 and the second amplifier 23 amplify input voltage and output the voltage. The first amplifier 12 amplifies input voltage to 0 to 60 V and outputs the voltage, while the second amplifier 23 amplifies input voltage to 0 to 30 V and outputs the voltage.

The first transformer 14, the second transformer 15 and the third transformer 24 convert (step-up or step-down) input voltage, and output the voltage. The first transformer 14 converts input voltage to 0 to 9000 V, and outputs the voltage, the second transformer 15 converts input voltage to 0 to 1600 V, and outputs the voltage, while the third transformer 24 converts input voltage to 0 to 30 V, and outputs the voltage.

The pulse wave generating circuit 21 generates a pulse wave of predetermined frequency in accordance with the voltage outputted by the third variable resistor 20.

The first connector 16 is a connector into which a plug of an electric bed 27 is inserted. The first sensor 18 connected to the first connector 16 detects that the plug of the electric bed 27 is inserted into the first connector 16, and sends a first detection signal to the control unit 2.

The second connector 17 is a connector into which a plug of a potential therapy probe 28 is inserted. The second sensor 19 connected to the second connector 17 detects that the plug of the potential therapy probe 28 is inserted into the second connector 17, and sends a second detection signal to the control unit 2.

The third connector 25 is a connector into which a plug of the low-frequency therapy probe 29 is inserted. The third sensor 26 connected to the third connector 25 detects that the plug of the low-frequency therapy probe 29 is inserted into the third connector 25, and sends a third detection signal to the control unit 2.

The first connector 16 is a connector dedicated to the electric bed 27, the second connector 17 is a connector dedicated to the potential therapy probe 28, and the third connector 25 is a connector dedicated to the low-frequency therapy probe 29. Therefore, when a plug of another probe is inserted into any of the first to third connectors 16, 17, 25, the first to third connectors 16, 17, 25 send to the control unit 2 an error signal to the effect that a plug of another probe has been inserted. For instance, when the plug of the low-frequency therapy probe 29 is inserted into the first connector 16, the first sensor 18 sends to the control unit 2 an error signal to the effect that another plug has been inserted.

The operating unit 3 is provided with a main power source switch, “high”, “medium” and “low” output level setting switches, a therapy time setting switch (5 to 20 minutes at 5-minute intervals), a therapy start/stop switch, and a switch for selecting between muscle stimulation therapy or nerve stimulation therapy during low-frequency therapy.

The first switch 13 is a switch for supplying current to the electric bed 27 or the potential therapy probe 28. When the first sensor 18 detects that the plug of the electric bed 27 is inserted into the first connector 16, and a first detection signal is sent to the control unit 2, the first switch 13 switches to the electric bed 27 in response to a control signal from the control unit 2 (CPU 201). When the second sensor 19 detects that the plug of the potential therapy probe 28 is inserted into the second connector 17 and a second detection signal is sent to the control unit 2, the first switch 13 switches to the potential therapy probe 28 in response to a control signal from the control unit 2 (CPU 201).

The second switch 22 is a switch for supplying low-frequency current to the low-frequency therapy probe 29, to carry out muscle stimulation therapy or nerve stimulation therapy. When the user selects muscle stimulation therapy by way of the operating unit 3, the second switch 22 switches to the second sine wave generator 9 and to the second variable resistor 11 (to muscle stimulation therapy) in response to a control signal from the control unit 2 (CPU 201). On the other hand, when the user selects nerve stimulation therapy by way of the operating unit 3, the second switch 22 switches to the third variable resistor 20 and to the pulse wave generating circuit 21 (to nerve stimulation therapy) in response to a control signal from the control unit 2 (CPU 201).

The electric bed 27 is for instance an electric bed having a conductor fabric provided within an insulating fabric, and having a high-voltage cord connected to an end of the conductor fabric via a terminal. The electric bed 27 is disposed, for instance, on the seat of a chair or underfoot. The potential therapy probe 28 comprises for instance a conductor, and has a high-voltage cord connected to an end of the conductor via a terminal. Potential can be applied locally to the treatment site by bringing the conductor into contact with the treatment site. The low-frequency therapy probe 29 comprises for instance an electrode provided in a pad having an adhesive surface, and has a high-voltage cord connected to an end of the electrode via a terminal. Pulse current (low-frequency current) can be applied to the treatment site by affixing the pad onto the treatment site.

The combined electric therapy apparatus according to the present embodiment has a power supply circuit not shown. The power supply circuit turns on and off a main power source of the combined electric therapy apparatus 1. When the switch of the main power source is switched on or off by way of the operating unit 3, a signal to the effect is sent to the CPU 201. The power supply circuit turns then on or off the main power source of the combined electric therapy apparatus 1 in response to a signal from the CPU 201.

The timer 4 is connected to the control unit 2 (CPU 201). The timer 4 has the function of timing the therapy time, which is set by way of the operating unit 3, on the basis of a control signal from the control unit 2 (CPU 201).

The receiving unit 5 has an infrared sensor that can receive infrared rays. The receiving unit 5 receives infrared signals emitted by the remote control 30 and sends a signal to the control unit 2 (CPU 201). As a result, operations such as main power source on/off, output level setting, therapy time setting, therapy start/stop and the like can be carried out using the remote control 30.

The sound IC 6 generates sound data on the basis of a control signal from the control unit 2 (CPU 201), and causes sound to be outputted from the speaker 61 on the basis of that sound data. For instance, the sound IC 6 outputs acoustically an indication message on the basis of an indication signal sent by the CPU 201.

The image IC 7 generates image data on the basis of a control signal from the control unit 2 (CPU 201), and causes an image to be displayed on the liquid crystal (LCD) 71 on the basis of that image data. For instance, the image IC 7 generates operation image data on the basis of an operation image generation signal from the CPU 201, and causes the operation image to be displayed on the liquid crystal display 71. Also, the image IC 7 generates indication image data on the basis of an indication image generation signal from the CPU 201, and causes the indication image to be displayed on the liquid crystal display 71.

The process operation of potential therapy using the electric bed 27 in the combined electric therapy apparatus 1 is explained next with reference to the flowchart illustrated in FIG. 3.

When the user switches on the main power source by way of the operating unit 3, a signal to the effect is sent by the operating unit 3 to the CPU 201. On the basis of a control signal from the CPU 201, the power supply circuit is fed power from an AC power supply, and turns on the main power source of the combined electric therapy apparatus 1.

When the user inserts the plug of the electric bed 27 into the first connector 16, the first sensor 18 detects that the plug of the electric bed 27 is inserted into the first connector 16, and sends a first detection signal to the CPU 201. The CPU 201 receives the first detection signal from the first sensor 18 (S101).

Upon receiving the first detection signal sent by the first sensor 18, the CPU 201 determines whether it is receiving a second detection signal or a third detection signal (S102). If the CPU 201 determines that it is receiving a second detection signal or a third detection signal (S102, Yes), the CPU 201 executes an error display process (S103).

As illustrated in FIG. 4, in the error display process the CPU 201 sends to the sound IC 6 an indication signal to the effect of outputting acoustically an error message, and sends an indication image generation signal, for displaying an error message as an image to the image IC 7 (S103-1).

The sound IC 6 generates sound data on the basis of the indication signal from the CPU 201, while the image IC 7 generates image data on the basis of the indication image generation signal from the CPU 201. On the basis of the generated sound data, the sound IC 6 causes an error message to be acoustically outputted through the speaker 61, while the image IC 7 causes an error message image to be displayed on the liquid crystal display 71, on the basis of the generated image data.

The CPU 201 determines whether it is receiving only one among the first detection signal, the second detection signal and the third detection signal (S103-2). When the user removes the plug of the potential therapy probe 28 or the plug of the low-frequency therapy probe 29 from the second connector 17 or the third connector 25, and only the plug of the electric bed 27 is inserted, the CPU 201 determines that it is receiving only the first detection signal (S103-2, Yes), and sends a signal for discontinuing sound output to the sound IC 6, and a signal for discontinuing image display to the image IC 7 (S103-3). The error display process ends therewith. Thus, the combined electric therapy apparatus 1 according to the present embodiment notifies an error message as sound and images, when a plug is inserted into two or more among the first to third connectors 16, 17, 25. This allows urging the user to select a therapy method while precluding therapy from being carried out unless there is inserted only one plug. Therapy can be carried out safely as a result.

Upon termination of the error display process (S103), or when in step S102 it is determined that neither the second detection signal nor the third detection signal are being received (S102, No), the CPU 201 switches the first switch 13 to the electric bed 27, and sends to the image IC 7 a signal for generating an operation image relating to potential therapy using the electric bed 27 (S104).

On the basis of the signal from the CPU 201, the image IC 7 generates data relating to an operation image, and causes the operation image to be displayed on the liquid crystal display 71 on the basis of the generated data relating to the operation image. This allows the user to carry out the setting operation relating to potential therapy using the electric bed 27.

The CPU 201 determines whether the output setting in the operating unit 3 is set to “high”, “medium” or “low” (S105). For instance, when the user sets the output setting in the operating unit 3 to “high”, the CPU 201 determines whether first output termination point information corresponding to the set output setting “high” is stored or not in the sub storage unit 203, on the basis of an output setting signal from the operating unit 3 (S106).

When first output termination point information corresponding to the output setting “high” is not stored in the sub storage unit 203 (S106, No), the CPU 201 reads a 1/f fluctuation conversion program stored in the main storage unit 202 and generates voltage control data corresponding to the output setting “high” in accordance with a 1/f fluctuation conversion program (S107). The voltage control data corresponding to the output setting “high” is data for varying the output voltage within a 0 to 9000 V range according to a 1/f fluctuation cycle. The CPU 201 stores the generated voltage control data in the sub storage unit 203 (S108).

When the user sets the output setting to “medium” or “low” by way of the operating unit 3, and first output termination point information corresponding to these output settings is not stored in the sub storage unit 203, the CPU 201 reads a 1/f fluctuation conversion program stored in the main storage unit 202, generates voltage control data corresponding to the output setting “medium” or “low” in accordance with a 1/f fluctuation conversion program (S107), and stores the voltage control data in the sub storage unit 203 (S108). The voltage control data corresponding to the output setting “medium” is data for varying the output voltage within a 0 to 7000 V range according to a 1/f fluctuation cycle. The voltage control data corresponding to the output setting “low” is data for varying the output voltage within a 0 to 5000 V range according to a 1/f fluctuation cycle.

Next, the CPU 201 generates time control data in which output time of voltage based on the generated voltage control data is converted to a 1/f fluctuation cycle in accordance with a 1/f fluctuation conversion program (S109), and stores the generated time control data, associated to the voltage control data, in the sub storage unit 203 (S110). Time control data is data for varying the time at which there is outputted voltage varying based on the voltage control data, within a range from 0.1 to 10 seconds, according to a 1/f fluctuation cycle.

When first output termination point information corresponding to the output setting “high” is stored in the sub storage unit 203 (S106, Yes), the CPU 201 reads the first output termination point information stored in the sub storage unit 203 (S111).

The CPU 201 determines whether a therapy time is set by the operating unit 3 (S112). When the user sets a therapy time by way of the operating unit 3, the CPU 201 stores the set therapy time in the sub storage unit 203 (S113). The CPU 201 determines then whether the therapy start switch is switched on in the operating unit 3 (S114).

When the user switches on the therapy start switch in the operating unit 3, the CPU 201 sends to the timer 4 a signal relating to the set therapy time (S115), and reads voltage control data and time control data stored in the sub storage unit 203 (S116).

The CPU 201 controls the first variable resistor 10 on the basis of the read voltage control data and time control data (S117). At this time, the CPU 201 controls the first variable resistor 10 in such a manner that voltage is increased and reduced gradually at a rate of 1000 V/0.5 seconds. Increasing and reducing voltage gradually allows reducing inrush currents and diminishing adverse effects on circuitry and the human body. Upon reading the first output termination point information in the above step S112, the CPU 201 controls the first variable resistor 10 on the basis of control data, from the output termination point in the voltage control data and the time control data onward, in accordance with the read first output termination point information (S117).

Through control of the first variable resistor 10 by the CPU 201, the output voltage generated at the secondary side of the first transformer 14 via the first amplifier 12 is controlled in such a manner that the output voltage varies based on a 1/f fluctuation cycle. The voltage generated in the first transformer 14 and varying according to a 1/f fluctuation cycle is outputted to the electric bed 27, whereupon the latter applies potential to the body.

Upon receiving a signal relating to therapy time termination from the activated timer 4 (S118), the CPU 201 discontinues voltage generation based on that signal (S119), terminating thereby the process operation, and stores information relating to the point at which voltage output is terminated (first output termination point information) in the sub storage unit 203 (S120).

Thus, the CPU 201 reads voltage control data and time control data, and, on the basis of these data, controls the first variable resistor 10, and therefore the first transformer 14, so that the generated voltage is outputted to the electric bed 27 until the set therapy time has elapsed. A potential varying according to a 1/f fluctuation cycle is applied to the user as a result. The combined electric therapy apparatus 1 according to the present embodiment prevents thus the user from getting acclimatized to potential stimulation, and allows sustaining the therapeutic effect while imparting a relaxing feeling to the user.

The operation process of low-frequency therapy using the low-frequency therapy probe 29 in the combined electric therapy apparatus 1 according to the present embodiment is explained next on the basis of the flowchart illustrated in FIG. 5.

When the user switches on the main power source via the operating unit 3, a signal to the effect is sent by the operating unit 3 to the CPU 201. On the basis of a control signal from the CPU 201, the power supply circuit is fed power from an AC power source, and turns on the main power source of the combined electric therapy apparatus 1.

When the user inserts the plug of the low-frequency therapy probe 29 into the third connector 25, the third sensor 26 detects that the plug of the low-frequency therapy probe 29 is inserted into the third connector 25, and sends a third detection signal to the CPU 201. The CPU 201 receives the third detection signal from the third sensor 26 (S201).

Upon receiving the third detection signal from the third sensor 26, the CPU 201 determines whether it is receiving a first detection signal or a second detection signal (S202). When the CPU 201 determines that it is receiving a first detection signal or a second detection signal (S202, Yes), the CPU 201 executes an error display process, as illustrated in FIG. 4 (S203).

Upon termination of the error display process (S203), or when in step S202 it is determined that neither a first detection signal nor a second detection signal are being received (S202, No), the CPU 201 sends to the image IC 7 a signal for generating operation image relating to low-frequency therapy using the low-frequency therapy probe 29 (S204).

On the basis of the operation image generation signal, the image IC 7 generates data relating to an operation image, and causes an operation image to be displayed on the liquid crystal display 71 based on the generated data relating to the operation image. Specifically, the liquid crystal display 71 displays an image for urging the user to select muscle stimulation therapy or nerve stimulation therapy.

When the user selects muscle stimulation therapy or nerve stimulation therapy by way of the operating unit 3, in accordance with the operation image displayed on the liquid crystal display 71, the CPU 201 receives from the operating unit 3 a signal to the effect that muscle stimulation therapy has been selected or a signal to the effect that nerve stimulation therapy has been selected (therapy method selection signal) (S205). On the basis of the therapy method selection signal, the CPU 201 switches the second switch 22 to the second variable resistor 11 (to muscle stimulation therapy) or to the pulse wave generating circuit 21 (to nerve stimulation therapy) (S206). For instance, when the user selects muscle stimulation therapy by way of the operating unit 3, the CPU 201 sends a control signal to the second switch 22 so as to switch the second switch 22 to the second variable resistor 11, on the basis of the therapy method selection signal from the operating unit 3, whereupon the second switch 22 is switched to the second variable resistor 11.

The CPU 201 receives from the operating unit 3 a signal relating to output setting (output setting signal), and determines whether the output setting in the operating unit 3 is set to “high”, “medium” or “low” (S207). For instance, when the user sets the output setting in the operating unit 3 to “high”, the CPU 201 determines whether second output termination point information corresponding to the set output setting “high” is stored or not in the sub storage unit 203, on the basis of an output setting signal from the operating unit 3 (S208).

When second output termination point information corresponding to the output setting “high” is not stored in the sub storage unit 203 (S208, No), the CPU 201 reads a 1/f fluctuation conversion program stored in the main storage unit 202 and generates pulse height control data corresponding to the output setting “high” in accordance with a 1/f fluctuation conversion program (S209). Pulse height control data corresponding to the output setting “high” is data for varying pulse height within a 0 to 30 V range according to a 1/f fluctuation cycle. The CPU 201 stores the generated pulse height control data in the sub storage unit 203 (S210).

When the user sets the output setting to “medium” or “low” by way of the operating unit 3, and second output termination point information corresponding to these output settings is not stored in the sub storage unit 203, the CPU 201 reads a 1/f fluctuation conversion program stored in the main storage unit 202, generates pulse height control data corresponding to the output setting “medium” or “low” in accordance with a 1/f fluctuation conversion program (S209), and stores the pulse height control data in the sub storage unit 203 (S210). Pulse height control data corresponding to the output setting “medium” is data for varying pulse height within a 0 to 15 V range based on 1/f fluctuation cycles, and pulse height control data corresponding to the output setting “low” is data for varying pulse height to within a 0 to 10 V range according to a 1/f fluctuation cycle.

Next, the CPU 201 generates pulse width control data in which pulse width is converted to a 1/f fluctuation cycle in accordance with a 1/f fluctuation conversion program (S211), associates the generated pulse width control data to the pulse height control data, and stores the data in the sub storage unit 203 (S212). Pulse width control data is data for varying the pulse width within a 2 to 20 ms range, according to a 1/f fluctuation cycle.

Further, the CPU 201 generates pause time control data in which pause time is converted to a 1/f fluctuation cycle in accordance with a 1/f fluctuation conversion program (S213), associates the generated pause time control data to pulse height control data and pulse width related data, and stores the data in the sub storage unit 203 (S214). Pause time control data is data for varying the pause time (time from pulse fall to pulse rise) within a 2 to 20 ms range, according to a 1/f fluctuation cycle.

When second output termination point information corresponding to the output setting “high” is stored in the sub storage unit 203 (S208, Yes), the CPU 201 reads the second output termination point information corresponding to the output setting “high”, which is stored in the sub storage unit 203 (S215).

The CPU 201 determines whether a therapy time is set via the operating unit 3 (S216). When the user sets a therapy time by way of the operating unit 3, the CPU 201 stores the set therapy time in the sub storage unit 203 (S217). The CPU 201 determines then whether the therapy start switch is switched on in the operating unit 3 (S218).

When the user switches on the therapy start switch in the operating unit 3, the CPU 201 sends to the timer 4 a signal relating to the set therapy time (S219), and reads pulse height control data, pulse width related data and pause time control data stored in the sub storage unit 203 (S220).

The CPU 201 controls the second variable resistor 11 (or controls the third variable resistor 20 when in step S205 above the CPU 201 receives a signal to the effect that nerve stimulation therapy is selected) on the basis of the read pulse height control data, pulse width control data and pause time control data (S221). Upon reading second output termination point information in step S215, the CPU 201, according to the second output termination point information, controls the second variable resistor 11 (or the third variable resistor 20) on the basis of control data from the second output termination point onward from among the pulse height control data, the pulse width control data and the pause time control data (S221).

The CPU 201 controls the second variable resistor 11 (or the third variable resistor 20) to control thereby, via the second amplifier 23, the pulse height, pulse width and pause time of the pulse voltage generated on the secondary side of the third transformer 24 in such a manner that the pulse height, pulse width and pause time vary according to a 1/f fluctuation cycle. Changing the pulse width and pause time according to a 1/f fluctuation cycle allows the pulse frequency to be changed also according to a 1/f fluctuation cycle. The pulse voltage generated at the third transformer 24 is outputted to the low-frequency therapy probe 29, whereupon the low-frequency therapy probe 29 applies to the body pulse current whose pulse height, pulse width and pause time vary according to a 1/f fluctuation cycle.

Upon receiving a signal relating to therapy time termination from the activated timer 4 (S222), the CPU 201 discontinues pulse voltage generation based on that signal (S223), terminating thereby the process operation, and stores information relating to the output termination point (second output termination point information) in the sub storage unit 203 (S224).

The above embodiment has been described for facilitating understanding of the present invention, and not for limiting the present invention. The various elements described in the above embodiment are thus deemed to also include all design modifications and equivalents falling under the technical scope of the present invention.

For instance, the combined electric therapy apparatus 1 according to the present embodiment may comprise a comparator circuit that measures the output voltage values of the first transformer 14, the second transformer 15 or the third transformer 24, the measurement results being then inputted into the control unit 2 (CPU 201) such that when the measurement results differ from set voltage values, the control unit 2 (CPU 201) controls the first variable resistor 10, the second variable resistor 11 or the third variable resistor 20 in such a manner that output voltage values take on the set voltage values.

The combined electric therapy apparatus 1 according to the present embodiment may comprise also a spark detector, such that when a spark is detected, information to the effect is sent to the control unit 2 (CPU 201), whereupon voltage output is discontinued on the basis of an instruction from the control unit 2 (CPU 201).

1. A potential therapy apparatus, comprising:
a voltage generating unit for generating a predetermined voltage;
a control unit for varying, according to a 1/f fluctuation cycle, a voltage generated by the voltage generating unit and varying output time of the voltage; and
a potential therapy conductor terminal for applying potential from the voltage generating unit to a therapy object.

2. The potential therapy apparatus according to claim 1, further comprising an output operation unit that can set an upper limit of the output voltage, wherein
the control unit causes the voltage to vary within the upper limit of the output voltage set by the output operation unit, according to a 1/f fluctuation cycle.

3. The potential therapy apparatus according to claim 1, wherein
the control unit comprises:
a storage unit in which voltage control data for varying the voltage generated by the voltage generating unit and time control data for varying the output time of the voltage generated by the voltage generating unit are associated with each other and then stored; and
a processing unit for varying the voltage generated by the voltage generating unit and varying the output time of the voltage according to a 1/f fluctuation cycle, based on voltage control data and time control data stored in the storage unit.

4. The potential therapy apparatus according to claim 3, further comprising:
a timer for controlling energization time; and
a timer operation unit that can set an energization time, wherein
the processing unit causes the voltage generated by the voltage generating unit, and the output time of the voltage, to vary according to a 1/f fluctuation cycle, until termination of the energization time set by the timer operation unit.

5. The potential therapy apparatus according to claim 4,
wherein, upon termination of the energization time set by the timer operation unit, the storage unit stores information relating to a termination point in the voltage control data and the time control data at the time at which the energization time terminates, and when information relating to the termination point is stored in the storage unit, the control unit causes the voltage generated by the voltage generating unit, and the output time of the voltage, to vary according to a 1/f fluctuation cycle, based on the voltage control data and the time control data from the termination point onward, in accordance with information relating to the termination point.

6. The potential therapy apparatus according to claim 1, wherein the control unit increases and reduces gradually the voltage generated by the voltage generating unit.

7. The potential therapy apparatus according to claim 1, further comprising: a plurality of connectors into which respective plugs of a plurality of the potential therapy conductor terminals are inserted; and a sensor that detects the insertion of the plugs into the plurality of connectors respectively and sends a detection signal to the control unit, and when the plugs are inserted into two or more connectors of the plurality of connectors, the control unit outputs an indication signal on the basis of a detection signal from the sensor.

8. A combined electric therapy apparatus, comprising:
a voltage generating unit for generating a predetermined voltage;
a pulse voltage generating unit for generating a predetermined pulse voltage;
a control unit for controlling the voltage generating unit and the pulse voltage generating unit;
a potential therapy conductor terminal for applying potential from the voltage generating unit to a therapy object; and
a low-frequency therapy conductor terminal for applying pulse current from the pulse voltage generating unit to a therapy object, wherein
the control unit causes the voltage generated by the voltage generating unit, and the output time of the voltage, to vary according to a 1/f fluctuation cycle, and causes the pulse height, pulse width and pause time of the pulse voltage generated by the pulse voltage generating unit to vary according to a 1/f fluctuation cycle.

9. The combined electric therapy apparatus according to claim 8, further comprising an output operation unit that can set an upper limit of output voltage or of output pulse voltage, wherein
the control unit causes, according to a 1/f fluctuation cycle, the voltage or pulse height to vary within the upper limit of output voltage or output pulse voltage set by the output operation unit.

10. The combined electric therapy apparatus according to claim 8, wherein
the control unit comprises:
a storage unit in which voltage control data for varying the voltage generated by the voltage generating unit, and time control data for varying the output time of the voltage generated by the voltage generating unit are associated with each other and then stored, and pulse height control data for varying pulse height of the pulse voltage generated by the pulse voltage generating unit, pulse width control data for varying the pulse width of the pulse voltage generated by the pulse voltage generating unit, and pause time control data for varying the pause time of the pulse voltage generated by the pulse voltage generating unit are associated with each other and then stored; and
a processing unit for varying the voltage generated by the voltage generating unit and varying the output time of the voltage according to a 1/f fluctuation cycle, based on voltage control data and time control data stored in the storage unit, and varying according to a 1/f fluctuation cycle the pulse height, pulse width and pause time of the pulse voltage generated by the pulse voltage generating unit on the basis of pulse height control data, pulse width control data and pause time control data stored in the storage unit.

11. The combined electric therapy apparatus according to claim 10, further comprising:
a timer for controlling energization time; and
a timer operation unit that can set an energization time, wherein
the processing unit causes the voltage generated by the voltage generating unit, and the output time of the voltage, to vary according to a 1/f fluctuation cycle, and causes the pulse height, pulse width and pause time of the pulse voltage generated by the pulse voltage generating unit to vary according to a 1/f fluctuation cycle, until termination of the energization time set by the timer operation unit.

12. The combined electric therapy apparatus according to claim 11, wherein
upon termination of the energization time set by the timer operation unit, the storage unit stores information relating to a first termination point in the voltage control data and the time control data at the time at which the energization time terminates, and stores information relating to a second termination point in the pulse height control data, the pulse width control data and the pause time control data at the time at which the energization time terminates,
when information relating to the first termination point is stored in the storage unit, the processing unit causes the voltage generated by the voltage generating unit, and the output time of the voltage, to vary according to a 1/f fluctuation cycle, based on the voltage control data and the time control data, from the first termination point onward, in accordance with information relating to the first termination point, and
when information relating to the second termination point is stored in the storage unit, the processing unit causes the pulse height, pulse width and pause time of the pulse voltage generated by the pulse voltage generating unit to vary according to a 1/f fluctuation cycle, based on the pulse height control data, the pulse width control data and the pause time control data, from the second termination point onward.

13. The combined electric therapy apparatus according to claim 8, wherein the control unit increases and reduces gradually the voltage generated by the voltage generating unit.

14. The combined electric therapy apparatus according to claim 8, further comprising:
a first connector into which a plug of the potential therapy conductor terminal can be inserted;
a second connector into which a plug of a low-frequency therapy conductor terminal can be inserted;
a first sensor for detecting that a plug of the potential therapy conductor terminal is inserted into the first connector, and for sending a first detection signal to the control unit; and
a second sensor for detecting that a plug of the low-frequency therapy conductor terminal is inserted into the second connector, and for sending a second detection signal to the control unit, wherein
upon receiving the first detection signal, the control unit controls the voltage generating unit, and upon receiving the second detection signal, the control unit controls the pulse voltage generating unit.

15. The combined electric therapy apparatus according to claim 14, further comprising a liquid crystal display, wherein
upon receiving the first detection signal or the second detection signal, the control unit displays on the liquid crystal display a therapy setting display image based on the first detection signal or second detection signal.

16. The combined electric therapy apparatus according to claim 14, wherein the control unit in a state of being receiving the first detection signal or the second detection signal outputs an indication signal upon receiving the second detection signal or the first detection signal.
